(19) United States
(12) Patent Application Publication
(10) Pub. No.: US 2013/0325059 A1
(43) Pub. Date: Dec. 5, 2013

(54) Non-Clogging Airless Spray for High Viscosity, High Surface Tension Fluids

(71) Applicant: William Gerald O'Neill, Maple Grove, MN (US)

(72) Inventor: William Gerald O'Neill, Maple Grove, MN (US)

(73) Assignee: DEVICEGENERICS, INC., Maple Grove, MN (US)

(21) Appl. No.: 13/486,852

(22) Filed: Jun. 1, 2012

(51) Int. Cl. A61B 17/03

(52) U.S. Cl. 606/213

(57) ABSTRACT

The invention describes a dispensing spray device coupled to a dual syringe device whose outlets terminate in a plurality of small holes of sufficient small size to induce high velocity jets in the fluid exiting each of the holes. The holes in two caps are forced into orientation with respect to each other at an angle governed by the included angle of the two caps. The two fluids exit in a plurality of discrete streams and combine in a shower pattern away from the caps. The liquids are preferably two components of a tissue sealant or tissue adhesive.

### TECHNICAL FIELD

The invention solves a problem for spraying and mixing two high viscosity fluids in medical applications. Specifically the invention is used for spraying a two component, reactive mixture for stopping bleeding during surgery.

### REFERENCES CITED

### U.S. Patent Documents

U.S. Pat. No. 5,639,025 June 1997 Bush et al.

U.S. Pat. No. 5,088,649 February 1992 Hanson et al.

U.S. Pat. No. 3,701478 October 1972 Tada et al.

U.S. Pat. No. 7,682,336 May 2005 Hoogenakker et al.

U.S. Pat. No. 5,605,255 February 1997 Reidel et al.

U.S. Pat. No. 6,461,325 Delmotte et al.

U.S. Pat. No. 6,835,186 December 2004 Pennington et al.

### Other Publications

O'Lenick, A. J. Comparatively speaking: Lowering Tension in Water vs. Oil, http://www.cosmeticsandtoiletries.com/research/methodsprocesses/99891669.html

Surface Tension Values of some Common Test Liquids for Surface Energy Analysis; http://www.surface-tension.de/

### Potter and Foss; Fluid Mechanics, John Wiley and Sons, 1975

### BACKGROUND

Bleeding is a common problem during many types of surgeries. There are a variety of methods of controlling bleeding including electrocautery, sponges or compressions, and biological hemostatic agents. Hemostasis is the science of controlling bleeding. All of these hemostatic agents have applications for controlling bleeding (hemostasis). Electrocautery is useful to cut through tissue since the electrocautery device both cuts and seals the wound. Sponges and compressions are often used and since the 1950's sponges have been made from bioabsorbable materials such as cellulosic, plant based fibers. Some bleeding problems do not lend themselves to cautery or sponges. If bleeding is coming from an anastomosis site (joining of two vessels such as bypassing an artery around a blockage during open heart surgery), it is not practical to apply electrocautery to these delicate arteries. Packing sponges around the wound may not be practical; the beating heart for example may dislodge sponges packed around a bypassed artery. During surgery on the kidney to remove a tumor, a relatively large surface area may be cut away and bleed. While electrocautery is used to seal the wounds, the bleeding is often still prevelant and fibrin sealants may be employed.

Several manufacturers make fibrin sealants which combine fibrinogen and thombin to start the clotting cascade. These hemostatic agents are highly effective at controlling bleeding from cut arteries and veins, surgical anastomosis sites and tissue removed during surgery. Baxter and Johnson and Johnson Ethicon both sell fibrin sealants under the brand names “Tisseel” and “Evicel” respectively. The two companies dominate the market for fibrin sealants. The human derived fibrinogen and thrombin are frozen when delivered to the hospital and must be thawed prior to use. When thawed and in a liquid state, the viscosity of these fluids ranges between 40 cP to over 300 cP. The tissue sealants can be used when the blood has anti-coagulation agents such as heparin. During open heart surgery, the blood is exposed to many foreign surfaces such as plastic tubing, catheters and oxygenators. To prevent blood from clotting in these devices, heparin is often administered to the patient. Blood with anti-coagulants can be difficult to control and fibrin sealants may help control bleeding in these applications.

Fibrin sealants are delivered by either dripping the two solutions on the wound or spraying the thrombin and fibrinogen onto the bleeding site. Dripping is simple but may be less desirable in some instances than spraying. When the two part solutions are dripped onto a large wound, it may be desirable to have a light, even coating of the two materials. This can be a challenge when the two liquids drip from the dispensing catheter. Another problem with dripping the two solutions is that the two solutions may combine at the tip and quickly clot prior to reaching the bleeding site. This problem of premature clotting clogs the tips of these dispensing applicators. Clogged tips in applicators may be cut away to reopen the two passages in a plastic cannula but this extra step slows the delivery of the hemostatic agent.

Spraying can be useful for applying a light, even coating over a large, diffuse surface. However spraying is useful in applying liquids which should not combine until well away from the dispensing tip such as fibrin sealants.

### Prior Methods for Spraying High Viscosity Liquids

The most common method used for spraying high viscosity fluids is to flow air over the dispensing device. Air supplied at a sufficient volumetric flow rate will atomize the droplets of the high viscosity liquids and provide a very uniform spray coating. This technology is similar to paint spraying nozzles which use compressed air to atomize the paint droplets and create a uniform coating on the surface on which paint is to be applied. Both Tisseel and Evicel make dispensing tips which accommodate an air source and spraying the sealants using these air driven applicators is very common. A pressure regulator is used to control the air pressure from the compressed air source in the operating room. Both Johnson and Johnson and Baxter sell regulators and tubing to connect the regulator to the air source and to the spray applicator.

Hoogenakker (U.S. Pat. No. 7,682,336) and Delmotte et al (U.S. Pat. No. 6,461,325) describes such a compressed air catheter for spraying biological materials to stop bleeding.

The drawback to using air driven spray systems is the added complexity of bringing a compressed air source to the sterile surgical site. If the surgeon suspects that bleeding may be a problem during a procedure, the operating room staff may prepare the fibrin sealant in advance. However if unexpected bleeding occurs, prepping the sealant and preparing the air regulator may be more time and effort than is desired by the surgeon. The surgeon may not know in advance the nature of the wound. If during the procedure it is desirable to spray the fibrin sealant, the procedure may be delayed as the o.r. staff finds and connects the regulator and tubing.

An additional safety risk with air driven spray systems is the risk of life threatening air embolism. On Oct. 5, 2009, FDA section of Vaccines, Blood and Biologics issued a notice to Baxter regarding it's Artiss fibrin sealant spray applicator. FDA required that Baxter relabel the product with warnings about a “life threatening risk or air or gas embolism with the use of spray devices employing a pressure regulator to administer fibrin sealants”. FDA received complaints that the use of the spray device at higher than recommended pressures and in close proximity to the surface of the tissue could result in air being introduced into a blood vessel. FDA mentioned in the notice that “This safety update applies to all fibrin sealants”.¹ The current invention prevents this risk by eliminating air as a delivery vehicle for the fibrin sealant. ¹ http://www.fda.gov/BiologicsBloodVaccines/SafetyAvailability/ucm209778.htm?utm campaign=Google2&utm source=fdaSearch&utm medium=website&utm term=fibrin%20sealant&utm content=10

Accelerants such as CFC's have been used to induce a spray pattern in paints and cooking sprays. These accelerants are not practical with sprays involving biological agents to stop bleeding in people. The regulatory pathway required to qualify spraying biological agents using a synthetic accelerant would be prohibitively long and expensive.

The ideal solution would be to have a dispensing tip which allowed for airless spray. Airless spray systems are fairly simple in single fluid, low viscosity applications. One technique used to create a low viscosity spray is to design the tip to force the fluid into a turbulent state. Another technique used to spray low viscosity fluids is to design a conical protrusion into the fluid path which causes the liquid to spread out into a conical fan. However a two part, high viscosity airless spray poses multiple challenges. To prevent clogging the two part components must be sprayed such that the two materials do not combine until well after exiting the tip. A second problem with airless spraying two component liquids is the difficulty in getting the viscous materials to form a spray pattern without the assist of air. Viscous fluids atomized particles or discrete fluid streams tend to combine into one fluid stream. Viscous fluids may be caused to disperse into a fan but will quickly recombine into a single stream.

A third problem with mixing high viscosity fluids is the high surface tension of fibrin sealants. Fibrin sealant surface tension is similar to that of a viscosity analogue; 76% glycerin and 24% water by weight. Oils such as vegetable oil have much lower surface tensions. Water and glycerine both have high surface tensions. Water has a surface tension of 72 dynes/cm. The surface tension of glycerine is 64 dynes/cm . According to O'Lenick by contrast, oil has a surface tension of 30-35 dynes/cm.

Water has a high surface tension as described below but is easily sprayed using multiple designs such as a cone in the flow field etc. The low viscosity of water (1 centipoise) helps the fluid overcome its relatively high surface tension. The low viscosity of water is conducive to transitioning the sprayed fluid from laminar flow to turbulent flow. The transition of liquids from laminar to turbulent flow in a tube is dictated by the dimensionless Reynolds number which is shown in equation 1 below. The viscosity of the fluid is found in the denominator. Hence a liquid with a viscosity of 40 cP will require 40 times the velocity to transition to turbulence than water at 1 cP.

Re=ρ*U*L/u Equation 1

Where ρ=density
U=velocity of the fluid
L=length of the pipe segment and
u is the viscosity

Bush (U.S. Pat. No. 5,639,025) describes a high viscosity pump sprayer utilizing a fan spray nozzle. Bush's nozzle uses an internal recess which allows fluid to flow into a “V” shaped channel. This method has advantages such as creating a fan spray. However the design was prototyped in the laboratory and failed to create an adequate spray pattern. A prototype was fabricated with the V shape described by Bush and attached to a small tube. A liquid with a viscosity of 40 centipoise (cP) was created by mixing 76% by weight glycerine and water. The fluid was delivered using a syringe. The fan spray simply recombined into one single fluid stream. The failure to produce the spray pattern as described by Bush when using identical geometry may be attributed to the high surface tension of the fluid analogue. Bush in contrast was attempting to spray vegetable oil. The relatively low surface tension of vegetable oil may allow for a spray pattern which may not be possible with the higher surface tensions fluids used in the experiment.

Hanson (U.S. Pat. No. 5,088,649) describes a pump spray nozzle diverting fluid into two high velocity streams which are force to converge. Hanson's converging stream method creates collisions of the streams to form fine drops. This invention is intended for hand pumping vegetable oil onto a cooking surface. This design was again prototyped in the laboratory with a tip forcing two high velocity streams to converge. The two fluid were forced down channels which had a width of 0.035 inches and a height of 0.001 inch. The very thin height was purposefully intended to accelerate the streams to a velocity close to turbulence. The result of the experimented did not yield a spray fan pattern but also induced a recombination of the two separate streams into one solid stream. The surface tension of the glycerine and water mixture was too high to prevent the atomized droplets from recombining. The converging stream spray concept may work acceptably for liquids such as cooking oil with a lower surface tension but does not work with liquids with higher surface tensions.

Tada (U.S. Pat. No. 3,701478) describes a hand sprayer with two thin elongated slots to induce a spray pattern. This design will also not work with high viscosity and high surface tension fluids for similar reasons; the high surface tension of the fluids will not permit divergence of the fluid stream into a fan.

Even if the problems of spraying high viscosity, high surface tensions could be easily overcome, spraying fibrin sealants introduces a third complexity; the two fluids are highly reactive and must be kept separated to prevent clogging of the device.

Reidel et al (U.S. Pat. No. 5,605,255) describes a fibrin sealant nozzle which combines the two components, fibrinogen and thrombin into one channel and sprays the combined fluid through a highly restrictive channel with a premixing chamber and an exit nozzle. The exit nozzle has impeller or fan shaped surfaces which induce a rotational velocity to the fluid prior to exiting a small hole. This concept suffers from a problem of combining the fluids prior to exiting the nozzle. This will cause the fluid to coagulate and the tip will clog if the user stops administering the fluid for more than 5-15 seconds. Reidel teaches that the tip may be exchanged if it clogs. However this may result in frequent tip changes which will be inconvenient and expensive since a complex new tip must be attached every time the tip clogs. The small channels in Reidel's invention lend themselves to premature clogging and the small, long channels can be restrictive to flow. The high resistance to flow of two viscous fluids will dramatically increase the force which the user must exert with his/her thumb on the dual plunger in order to expel the two fluids.

Pennington et al (U.S. Pat. No. 6,835,186) shows a fibrin sealant airless spray system which uses two spin chambers which spin the fluid. This device introduces a device which depends upon motion between the spinning impeller and the fluid chambers. Due to the high surface tension and viscosity combined with the potential for cross contamination between chambers, this spinning impeller may be impractical due to its tendency to freeze up.

The art of spraying high viscosity fluids without air appears to not solve the problems with higher surface tension fluids also exhibiting high viscosity. All of the examined prior art shows inventions which will spray high viscosity liquids without air but none describe spraying a liquid with both high viscosity and high surface tension without clogging upon cessation of fluid delivery. Hence there remains a need to solve the problem of creating a spray pattern for high surface tension and viscous liquids which does not suffer from frequent clogging.

### SUMMARY OF THE INVENTION

The present invention is directed to an improved hand pump, airless spray system for delivering two high viscosity and high surface tension fluids. The fluids being dispensed have a viscosity between 30-60 centipoise and a surface tension greater than 60 dynes/cm. The fluids are transferred into dual syringe syringe to which is attached the dispensing spray tip.

The invention is intended to dispense biological agents such as fibrin components onto a bleeding surface without the need for an external compressed air or any external energy source. The invention is designed to allow the user to dispense the two liquids comfortably with one hand. The invention comprises a dual syringe device containing the first and second liquids, a spray tip having a proximal and distal end. The syringes are adapted to be removably coupled to the proximal end of applicator spray tip to provide fluid communication to the spray nozzle located at the distal end of the spray tip. The applicator spray tip is comprised of a series of small holes which orient two fluids into a series of high velocity streams. The high velocity streams combine away from the exit holes to prevent tip clogging and to promote mixing.

### DESCRIPTION OF THE DRAWINGS

The forgoing features, objects and advantages of the present invention will become apparent to those skilled in the art from the following detailed description of the preferred embodiment.

FIG. 1 shows the dual syringe device coupled to the detachable spray tip used to contain and spray the two high viscosity and surface tension fluids.

FIG. 2 illustrates the spray exiting the small holes in the molded caps and combining at a controlled distance away from the caps.

FIG. 3 is a cross sectional view of one of the two attached molded caps and tubing. The view shows two of the multiple holes in profile.

FIG. 4 is an exploded view of the two molded caps and the two extruded flexible tubes. The exploded view shows the two surfaces which, when bonded together, keep the tips at a controlled inclusive angle.

FIG. 5 is a cross sectional view which illustrates the tongue and groove features which control the angle at which the tips point.

FIG. 6 is a cross sectional view of an alternative embodiment which results in a vena contracta to artificially decrease the effective cross sectional area of the exit holes

### DESCRIPTION OF THE PREFERRED EMBODIMENT

The object of this invention is to provide a delivery device capable of spraying two highly viscous fluids with high surface tension without frequent clogging of the tip. The two fluids are preferably the components of a two component sealant, in particular a fibrinogen based tissue adhesive.

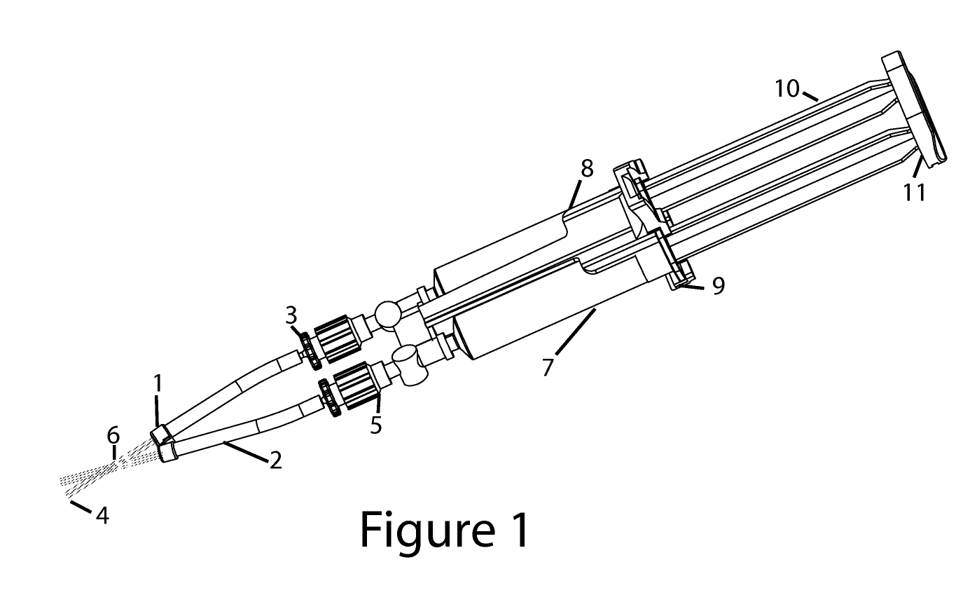

The two liquids are kept separated in two standard syringes (FIG. 1 items 7 and 8) coupled together by a plunger mechanism 11. The dispensing spray tip shown in FIG. 1 is connected to the dual syringe plunger by two mating luer connectors FIG. 1 items 3 and 5. The user would spray both fluids by placing two fingers into the applicator handle 9 while simultaneously placing a thumb on the plunger conector 11. By depressing the plunger mechanism 11, the two fluids are pushed out of the dual syringe applicator into two tubes (connecting piece)s 2 as shown in FIG. 1. The flexible connecting pieces or tubes 2 are bonded to an injection molded cap 1. Two identical caps are bonded together at a prescribed angle such that the fluid streams 4 exit both caps and combines at a location distal to both caps 6. This invention keeps the two liquids from contaminating each other and thereby clogging the delivery cap.

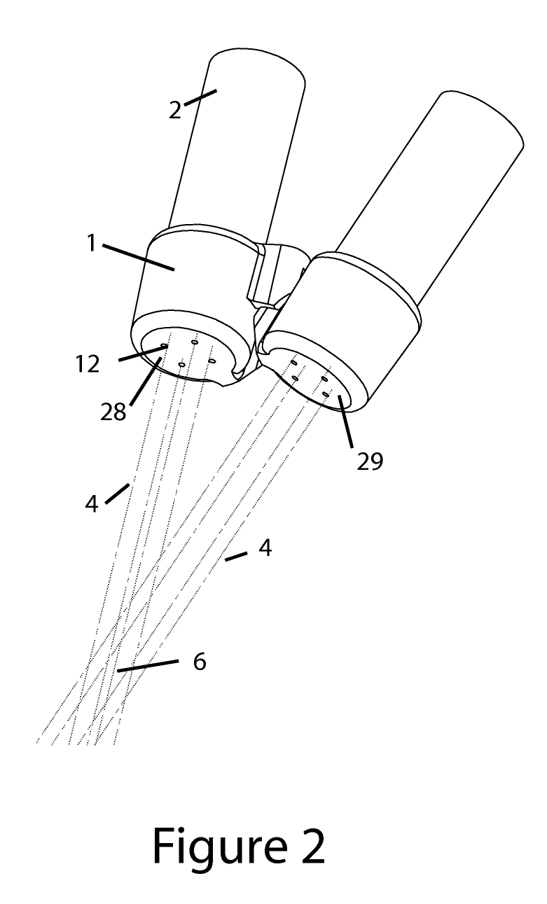

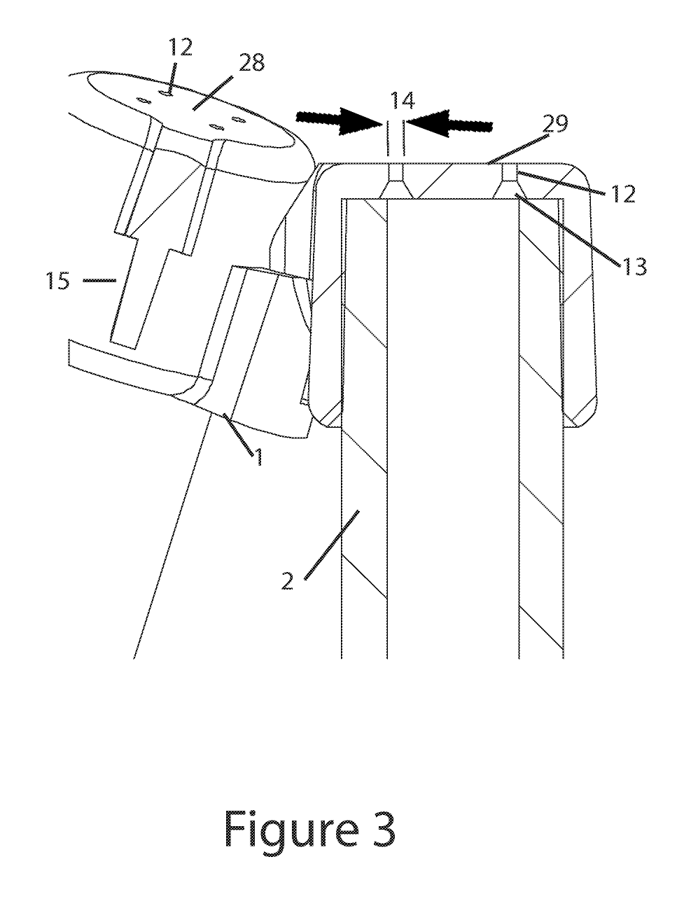

FIG. 2 shows a close up of the dispensing spray device. FIG. 2 illustrates the two connecting or tubes 2 rigidly attached to the molded caps 1. The ideal spray device creates a number of independent, high velocity streams which combine at minimum 1 cm from the exit of each cap. The high velocity is important such that the fluid exits and streams away from the cap. To prevent clogging, the two biologics are kept separate in each of the two tubes/caps and exit through a plurality of small holes 12. FIG. 3 shows a cross section of the tube and cap and shows two holes. The plurality of holes ranges from four to 15 individual holes ranging in size between 0.001 and 0.015 inch. The preferred spacing of the holes is at minimum 0.018 inches from each other. If the holes are spaced too closely, the exiting fluid streams will combine into a single, stream due to the high surface tension of the two liquids. If the hole diameter (FIG. 3 item 14) is greater than 0.015, there is insufficient fluid velocity and the fluid dribbles from the each hole 12. Larger holes are easier to manufacture cheaply. Small holes measuring 0.008 to 0.010 inch in diameter may be injection molded into the tips 1. Smaller holes may be laser drilled into thin plastic parts in diameters as small as 0.001 inch. FIG. 3 shows a series of molded holes with a beveled leading edge 13 leading into a straight section 12. The bevel 13 stiffens the core pin of the molded holes and increases the reliability of the mold. Hole diameters (14) smaller than 0.008 inch may be mechanically or laser drilled into the molded tips as a secondary operation or alternatively heat formed into the thermoplastic. The preferred embodiment is a minimum of at least 12 discrete holes in each molded tip (12 holes total) with diameters of no greater than 0.004 inch and spacing from one another of at least 0.018 inch. If the holes are too few and too small in diameter, the back pressure may be excessive for comfortable expression of the two fluids. If smaller diameter holes are employed, a higher number of holes is recommended. If the holes are too large, excessive fluid shall be ejected with little force applied to the plunger 11. Thus there is a balance between size and number of holes.

A higher number of holes of very small diameter is advantageous since it will increase the number of fluid streams and decrease the likelihood of the streams converging with a stream from the mating fluid. A higher number of streams will thereby increase the mixing of the two fluids. In practice, the instructions for use of the shower head spray device described in this invention should advise the user to wave the spray pattern back and forth over the wound. This waving of the spray dispenser would induce better, more homogenous mixing of the two fluids rather than completely relying on the individual fluid jets to combine and mix.

Decreasing the diameter of the small holes while increasing the number of holes helps limit the volume of biological material from being expressed from the dual syringe system. In experiments to reduce the idea to practice, at least eight (8) holes of diameter 0.003 inches diameter and preferably 12-15 holes gives a nice gentle spray pattern. Hole sizes above 0.008 inches diameter dispense an excessive amount of biological material into the surgical site.

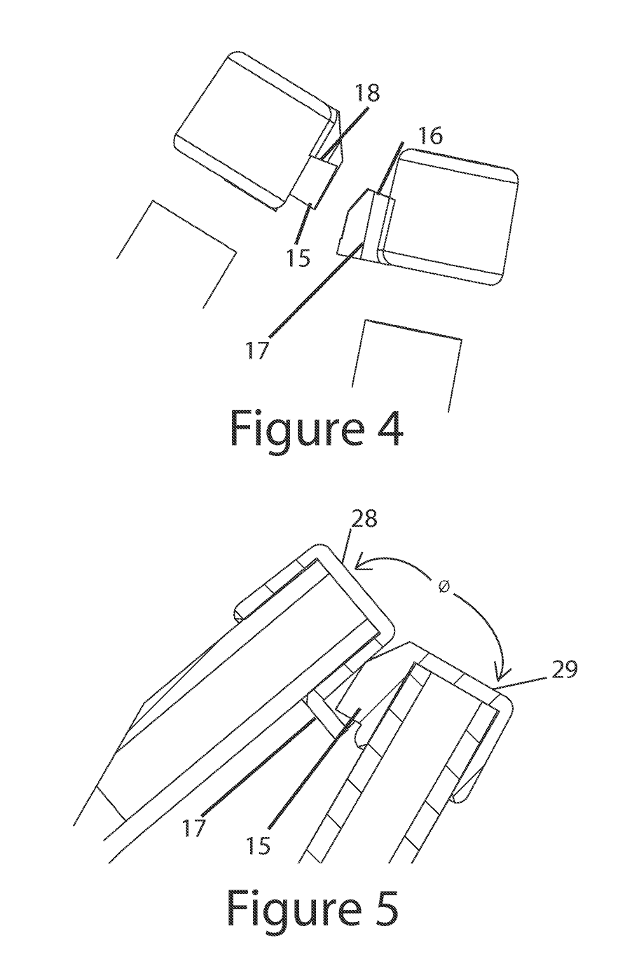

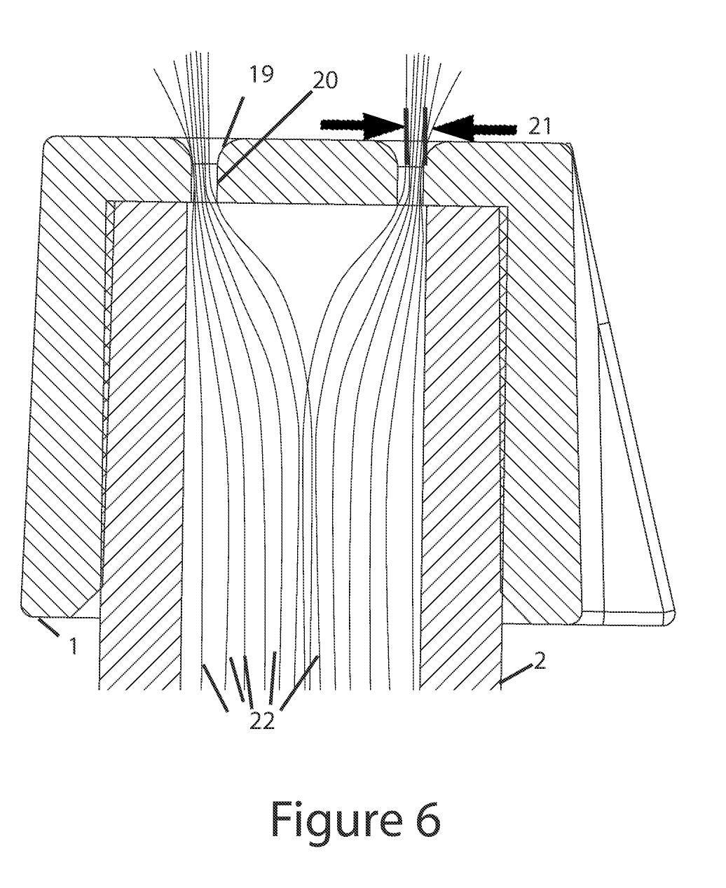

The caps are designed to mate such that the cap exit surfaces are angled from one another at a controlled inclination. FIGS. 4 and 5 show how the caps have mating features which, when bonded together, create an inclined angle theta (θ). The caps are designed to be interchangeable so one injection mold can produce both capsRegarding FIG. 4, each cap 1 contains a tongue 15 and groove 17. The tongue member 15 of the first cap slides easily into the groove slot 17 of the mating cap. At the top of both tongue 15 and groove 17 are two flat surfaces 16 and 17 which have a molded angle. In production, the operator would apply adhesive to the joint (either at location 15 or 17) and press the tongue into the groove while allowing the adhesive to rigidly form and connect the two caps. UV cure adhesive such as Loctite 3311 has been found to create a rapid joint between the two caps. The angle of surfaces 16 and 18 (FIG. 4) determine the angle theta (θ). The two fluids exit surfaces 28 and 29 as shown in FIG. 5 are thereby forced into an identical angle theta. Alternatively the two caps may be molded as single piece.

The inclusive angle θ should range between 160 degrees and 180 degrees between surfaces 28 and 29. The angle will determine the distance from the cap at which the two liquids streams will combine. A larger angle θ will create a combination location (FIG. 1 item 6) farther from the caps than a larger angle. A 180 degree angle implies the two cap surfaces are parallel and the streams will not converge. This may be a valid option in a minimally invasive laparoscopic application the two streams can be combined by moving the tip such that the two fluids overlap.

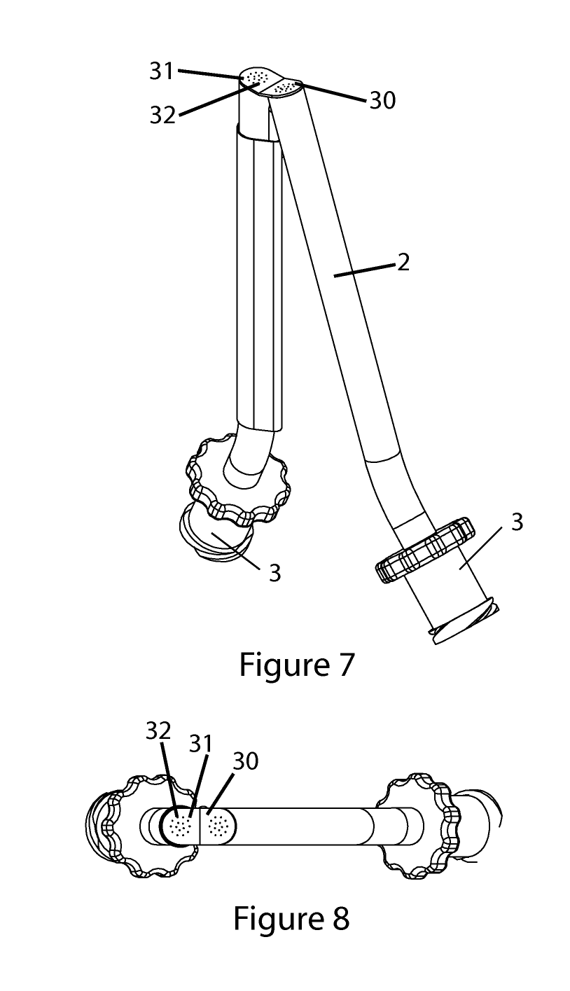

A second embodiment of the invention is shown in FIG. 6. In this embodiment, a radius 19 is applied to the exit hole 20. As opposed to FIG. 3 in which a chamfer is included on the inside of the cap, in FIG. 6, the radius is applied to the outside of the cap. The advantage of this design is that it create a dispersive spray in which the fluid streams flow away from each other and thereby create a wider spray pattern. A second advantage to the design is that this hole configuration creates an artificially smaller hole diameter and thereby creates a much higher exit velocity. As shown in FIG. 6, the biological fluid in each cap form streamlines of fluid flow. In FIG. 6, the streamlines are illustrated as items 22. The streamlines 22 are forced into one of the exit holes which are located to the outer edge of the inner surface of the tubing 2. Locating the holes as far away from the centerline of the tubing, will increase the distance between holes and thereby prevent the exiting fluid streams from combining into a single stream. The streamlines closest to the center of each tube must curve more than the streamlines in line with the hole 20. This causes a phenomenon known in fluid mechanics as a vena contracta or a necking down of a fluid stream. The vena contracta is illustrated in FIG. 6 as diameter 21. The diameter 21 is smaller than the actual measured inner diameter of each hole 20. As more fluid must pass through smaller holes, the result is an increase in the average velocity of each hole. Thus using this technique, molding holes as so illustrated in FIG. 6 shall create the effect of smaller holes with a larger, easier to mold core pin which will form the hole 20. This design makes it more practical to mold small holes since thicker core pins are less flimsy and are less likely to deflect due to the higher pressures common in injection mold cavities.

A third embodiment of the design is to combine the two tubes (connecting pieces) 2 into one dual lumen tube which is terminated by one molded spray cap 1. By leaving a slight gap between the end of the cut dual lumen tube and the spray holes, the two liquids may combine and create more homogenous fluid mixture. This third embodiment may improve the mixing of the two fluids at the expense of causing more frequent clogging of the caps.

A fourth embodiment of the design is illustrated in FIG. 7 and FIG. 8. In this case the exit holes 32 are drilled in a thin plastic film 33 which is bonded to the two connecting pieces 2. The plastic caps 1 described in FIGS. 1-6 are replaced by a single thin film 33. The plastic film 33 is angled via thermoforming or injection molding to form a controlled angle theta which ranges between 160 degrees and 180 degrees. This angle theta creates an included angle between face 30 and face 31. This embodiments allows very small holes (<0.004 inch) to be laser drilled through the plastic. A plurality of small holes 32 creates an improved shower pattern and promotes better mixing of the two fluids. However laser drilling of the exit holes requires relatively thin substrates 33. The ideal thickness of a substrate should be less than 0.015 inch and ideally the thickness of the substrate 33 is between 0.005 and 0.010 inch. This thin section becomes very difficult to injection mold but may be extruded or cast into a thin film. The substrate 33 can be adhesively bonded to the tubing 2 prior to laser drilling the holes.

We claim:

1. A medical device for spraying two liquids comprised of a first and second syringe each syringe having an outlet for a first and second liquid;
A connecting piece having first and second channels in communication with said syringe outlets terminating in distal component comprised of a spray cap which contain independent fluid passages for said first and second liquids and a first and second exit surface;
wherein first and second exit surfaces of said spray cap contain a plurality of small exit apertures and said first and second exit apertures create a spray pattern which combines and mixes said first and second liquids away from the device.

2. The medical device as described by claim 1 where the connecting channels and dispensing spray cap are combined into a spray tip which is removably attached to the dual syringes

3. The medical device as described by claim 1 where the fluid delivered is a tissue adhesive.

4. The medical device as described by claim 1 where the first and second liquids are a first and second component of an adhesive which become activated upon mixing

5. The medical device as described by claim 1 where the at least one of the two liquids have higher viscosity and higher surface tension than water or oil

7. The medical device as described by claim 1 where the angle between the first and second exit surfaces is between 160 and 180 degrees

8. A spray tip comprised of two connectors in fluid communication with a connecting piece having a first and second connecting channel and a distal spray cap containing two fluid channels;
Wherein the spray cap contains a first and second exit surface in fluid communication with the first and second connecting channel and said surfaces embody a plurality of small exit holes which permit the two liquids to combine and mix away from the spray tip
